(19) United States
(12) Patent Application Publication
(10) Pub. No.: US 2005/0278831 A1
(43) Pub. Date: Dec. 22, 2005

(54) Garment for enhancing male sexual performance

(76) Inventor: Daryl L. Dahlquist, Kent, WA (US)

Correspondence Address: Daryl Leroy Dahlquist, 15238 SE 272nd St, Kent, WA, 98042, US

(21) Appl. No.: 11/147,693

(22) Filed: Jun. 8, 2005

Related U.S. Application Data
(60) Provisional application No. 60/580,115, filed on Jun. 16, 2004.

(51) Int. Cl. A41B 9/02; A61F 5/00

(52) U.S. Cl. 2/403; 600/38

(57) ABSTRACT

A brief type undergarment is provided with an aperture for receiving the penis and testicles. The undergarment then mechanically supports an erection, but also acts on either the blood supply or nerves affecting the maintenance or an erection.

### RELATED APPLICATIONS

Provisional application No. 60,580,115 “Garment for Enhancing Male Sexual Performance”, filed June 2004

### U.S. GOVERNMENT INTEREST IN THE INVENTION

None

### BACKGROUND OF THE INVENTION

1. Field of the Invention

Therapeutic garments and appliances.

2. Description of Related Art

U.S. Pat. No. 6,027,464, “Sleeping and Therapy System with a Person Hydraulically Supported by Immersion in Water” by the present inventor describes a similar garment (FIG. 7) shows a similar garment as part of the support for a urinal system for use in the said sleeping system. The hole 38 shown is adapted to receive the penis and urinal assembly. The present invention receives both the penis and testicles and has an effect on sexual function, which is not supported by the prior art garment.

Several other patents utilizing a hole for receiving a penis have been located. Most, are for treating incontinence some other medical condition. One, U.S. Pat. No. 6,047,408 has a band across the top of the base of the penis for applying pressure to the upper side of the base of the penis. This is pressure is described as restricting blood flow out of a penis to enhance erection. The present invention does not apply a pinching action to any portion of the penis.

Only one patent, U.S. Pat. No. 6,553,266, by Jul-Jyabg Young, was found that had a hole adapted to receive both the penis and scrotum. This patent requires both to be unsupported in order to cause the garment to closely fit against the perineum region for the purpose of keeping an electrode in contact with the skin of the perineal floor. Mr. Young's mechanical structure of the garment is directed entirely to holding the necessary elements for electro-shock therapy of the peroneal muscles in both men and women. The hole is shaped only sufficiently large to pass the penis and scrotum of a male user, no pinching or holding.

3. Objects of the Invention

It is an object of the invention to provide mechanical support, and apply pressure on nerves, blood vessels, or perineal muscles for the purpose of enhancing sexual performance, in particular, assisting in maintaining an erection.

It is another object of the invention to serve as a substitute for pharmaceutical agents such as Viagra™, which are known to have serious side effects.

### BRIEF SUMMARY OF THE INVENTION:

A brief style undergarment constructed of elastic two-way stretch fabric such as “Lycra” or similar such as used in swimwear and athletic wear. The garment is equipped with an egg-shaped hole approximately 2¼ inches long, and preferably having an upper diameter 1⅕ inches across and a lower diameter approximately 1 inch across and, placed so that the penis and scrotum of a wearer may protrude without being pinched anywhere.

The cut (construction pattern) is such that the crotch panel snugs up against the perineal surface thereby applying an augmenting force to the perineal muscles and thence against the root structure of the penis in that region. In addition, other elastic panels surrounding the hole apply gentle pressures to the pubic area, and the zones alongside the penis and scrotum. Together, these forces enhance and prolong an erection.

Because the action of any one such garment is affected by the cut relative to the anatomy of the wearer, the hole position may need to be custom placed to fit the many body sizes and specific anatomical details of an individual or population.

To overcome this, in part, an embodiment with a separate detachable/movable hole piece is anticipated. The garment is equipped with an oversized oval opening with the long axis being vertical. The separate hole piece is attached by any of several means, hook-and-loop (Velcro) is preferred. This covers the upper and lower zones of the larger oval hole, and effectively causes the associated panels to function as a continuous panel with a properly positioned functional hole for admitting the penis and scrotum.

FIG. 2 shows another means to provide a variable size hole meeting the requirements of the invention. The upper and/or lower portions of an over-elongated oval can be closed by bands traversing the excess opening and fastened by Velcro, pins, lacing, etc.

Referring to FIGS. 3 and 4, in addition to the hole, the garment has another innovative feature where the back of the garment is significantly higher 10 than at the front. This allows the elastic waist band to fit properly between the small of the back and the area just under the abdominal “pot” and more importantly for those with a “pouch” of fat between the abdomen and the pubic area, the waist band rides on the pubic area, thereby fitting more closely to the skin of the wearer with no wrinkling from gathering of excessive fabric in the usually cut underwear. The band also provides a firm anchorage for the ends of the panel containing the hole. The hole panel is anchored at the front end directly to the waist band, and at the other end through the crotch thence to the back of the waist band, and permitting the garment to fit snugly and apply pressure to the perineal area and muscle.

### A BRIEF DESCRIPTION OF THE FIGURES

FIG. 1 shows the basic garment.

FIG. 2 shows an embodiment with an oval hole an a typical closing strap for closing the excess gap of the oval.

FIG. 3 shows the high-cut back of the present invention.

FIG. 4 shows the placement of the waist band on a user.

FIG. 5 shows an embodiment of the present invention having legs.

### INDEX OF IDENTIFIED ELEMENTS

1. The invention
2. The hole
3. Upper arc
4. Lower arc
5. Adjustment band
6. Elastic waist band
7. Fat paunch.
8. The usual position for underwear and trouser waist band.
9. The preferred position of the waist band for the present invention.
10. High cut of back of the present invention.
11. Leg part of an alternative embodiment of the present invention.

### DETAILED DESCRIPTION OF THE INVENTION

The garment of the present invention is preferably constructed of elastic material such as is use in swimwear or athletic wear (Lycra), although ordinary cotton knit has been found adequate for some users.

Because there is no need for providing room for the male external organs, the pattern for cutting can be essentially the same as for women's swimwear with modifications to adapt to the male torso such as thicker waist, male abdominal shapes, etc. The pubic and perineal panels should fit snugly against the body. In conventional brief style underwear with the penis and scrotum being inside the garment, the crotch panel cannot stretch snugly over the perineum surface, nor can the pubic panel be snugged against the pubes.

The preferred penis/scrotum hole 2 is an oval with the upper arc 3 having a diameter of approximately 1½ inches and the lower arc 4 having a diameter of approximately 1 inch. The length of the preferred hole is approximately 2½ inches. Like any garment, the positioning and the specific size of the hole and other garment parts is adjusted to match the body size of the wearer. The shape of the hole should be as described, but variations including an oval shape with both the upper and lower arcs the same size will work reasonably well, as will intermediate shapes and smaller arc diameters. In no case should the hole be so small or misplaced as to pinch the sides or top of the penis, or to bind at the underside of the scrotum. If the garment is cut on the pattern of some brands of brief style underwear, the top of the hole is approximately on the level as the top of the leg holes. Of course, leg hole styles vary considerably and this instruction should not be taken as a universal specification.

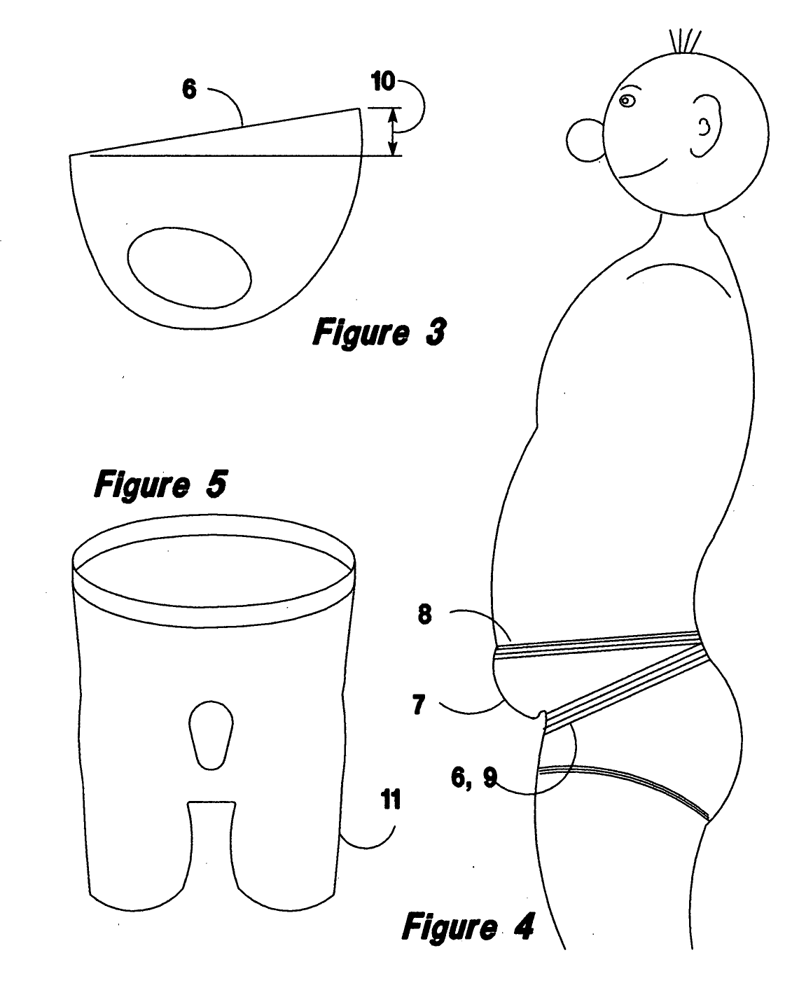

A second feature of the invention is that it can be specially cut for the man with an enlarged abdomen, ie a “pot”, and more importantly a man with a pouch of fat 7 hanging below the abdomen. Referring to FIG. 3, the back of the garment is cut higher 10 than the front by 1½ to 3 inches or more so that the waist band fits into the small of the back and around the waist to the upper pubic area. FIG. 4 shows the conventional waist band location 8 and the location used by the present invention 9. Conventional underwear and trousers are intended to be worn with the waistbands in the location identified as 8. But for the portly wearer, the conventional band will slip down below the abdomen which causes blousing of the extra fabric. For male users, the present invention requires the pubic panel to be snugged against the body, thus the gathering of the extra fabric interferes with the function of the invention.

The preferred fabric for use in the present invention is two-way stretch elastic material such as is used for swimsuits, athletic wear, and other snug-fitting garments, Lycra or similar.

How to Use the Invention:

The garment is pulled on over the buttocks and pelvis, and the penis and scrotum is pulled through the hole. The effect is to prolong erection.

Alternative Embodiments And Usages

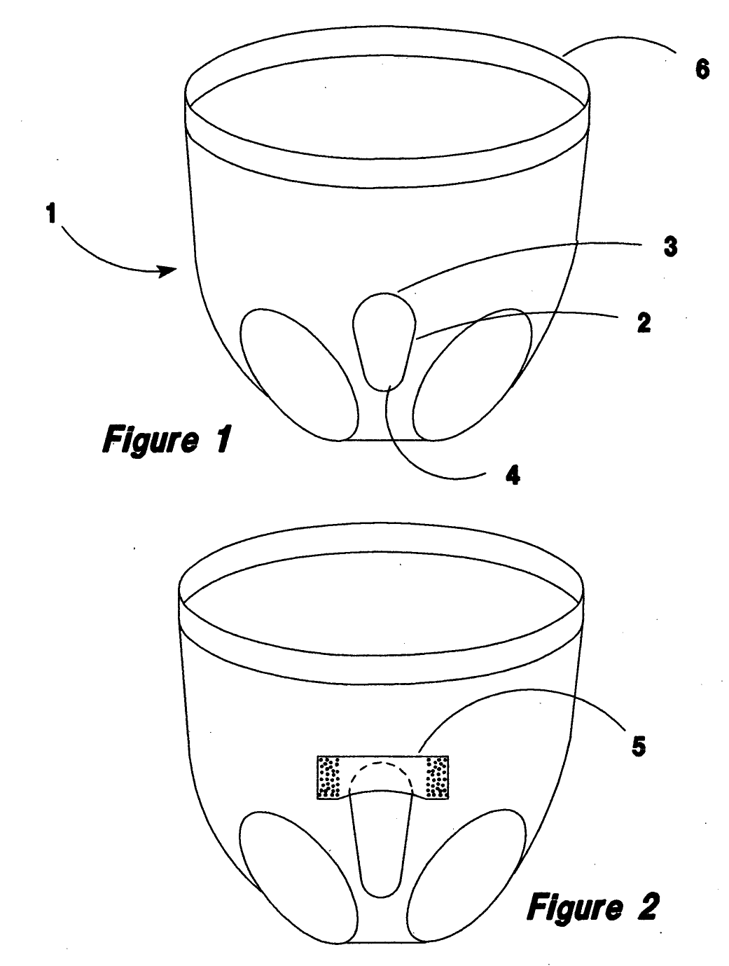

The brief design shown in FIG. 1 can be modified by adding legs 11 as shown in FIG. 5 for both the preferred usage, and the comfort-sleep wear described below.

The garment can be modified for enhancing comfort while sleeping, and in particular will provide a cooling effect during hot, humid nights, although the comfort feature is useful even on cool nights. A slight modification of the described invention is needed. The modifications are to have short legs 11 as shown in FIG. 5, be made of cotton or similar absorbent fabric, and optionally, a slightly larger hole. This embodiment puts a layer of cloth between the penis and scrotum and the leg during sleeping. Thereby reducing sweating and absorbing whatever sweat does occur at the contact area. The fabric not only controls sweating, but because of the contact area being dryer, the growth of fungus and bacteria is inhibited or eliminated. The cooling effect should also be healthier for the testes, which are sensitive to heat. Overheated testes reduces male fertility.

The sleep-comfort garment need not be closely conforming to the wearer. Boxer-style underwear and loose pajama wear will be adequate and still maintain a layer of fabric between the sleeper's leg and scrotum/penis.

The high back feature of the present invention can be used on trousers, skirts, ordinary underwear, and maternity clothing. The high back cut is particularly useful for portly men and women, especially those with a hanging pouch of fat under the abdomen. In general, garments with the high back/under the abdomen waist band (6 in FIG. 4) will result in a smoother better “put together” appearance and dramatically reduce the incidence of “plumber's exposure” by workmen bending over their work. The description of the waist band thus far has been with the band being made of elastic material to hold the garment in place. However, garments with the high back/under the abdomen waist can be secured to the wearer also by belts, or suspenders.

Another feature of the high back/under the abdomen waist band is that the position shown as 9 in FIG. 4 is a smaller circumference than the ordinary position identified in FIG. 4 as 8. In fact 9 is the shortest circumference of any waist measurement of a portly person. This means that an elastic in position 9 will have less hoop tension than other positions of wearing, and that the propensity of an elastic waist band or belt to slip down is greatly reduced.

1. A garment for enhancing male sexual performance comprising:
a. a close fitting garment having a hole adapted to receive the penis and scrotum of a male wearer protruding therethrough, and
b. said garment further comprising a panel adapted to conform to the perineal surface of the wearer, and
c. said garment further comprising a panel adapted to conform to the pubic area of the wearer.

2. The garment of claim 1 is constructed of primarily of elastic fabric, and where said conforming panels apply pressure to said areas.

3. The garment of claim 1 is constructed of moisture absorbing fabric.

4. The garment of claim 1 where said hole is oval shaped.

5. The garment of claim 1 where said hole is ovoid shaped and oriented with the small end downward.

6. The garment of claim 1 further comprising an elastic waist band adapted to encircle the wearer's torso where the back portion of the band traverses the small of the wearer's back and the front portion of the band traverses the wearer's pubic area.

7. A sleepwear garment for improving the comfort of a sleeping male person comprising:
a. a garment having a hole adapted to receive the penis and scrotum of a male wearer protruding therethrough, and
b. said garment further comprising a portion adapted to surround and cover the pelvic area of a person, and
c. said garment further comprising portions adapted to surround and cover the upper thigh portions of the legs of a person
d. where when said penis and scrotum are in place extending through said hole, said penis and scrotum are separated from direct contact with the skin of the wearer by the interposition of said upper thigh covering portions therebetween.

9. The garment of claim 8 is constructed of moisture absorbing fabric.

10. The garment of claim 1 further comprising an elastic waist band adapted to encircle the wearer's torso where the back portion of the band traverses the small of the wearer's back and the front portion of the band traverses the wearer's pubic area.

11. A garment adapted to cover at least the pelvic area of a human wearer's body comprising:
a. a garment having an upper end terminating in a band adapted to encircle the wearer's torso, and
b. said band having a portion adapted to traverse the small of the wearer's back, and
c. said band having a portion adapted to traverse the wearer's pubic area, and
d. when said garment is worn, the waist band encircles the wearer and extending from the small of the back thence around to the pubic area.

12. The garment of claim 11 where the garment is selected from the group comprised of trousers, sleepwear, skirts, and underwear.

13. The garment of claim 11 is removably attached to the wearer by at least one of the support means selected from the group consisting of an elasticized waistband, belt, and suspenders.
